(19) United States
(12) Patent Application Publication
(10) Pub. No.: US 2025/0360105 A1
(43) Pub. Date: Nov. 27, 2025

(54) USE OF ERGOTHIONEINE FOR ANTI-AGING

(71) Applicant: NANJING NUTRABUILDING BIO-TECH CO., LTD., Nanjing (CN)

(72) Inventors: Ronghua YI, Nanjing (CN); Shawn Wells, Nanjing (CN); Kylin LIAO, Nanjing (CN)

(21) Appl. No.: 19/200,191

(22) Filed: May 6, 2025

Related U.S. Application Data
(63) Continuation of application No. PCT/CN2023/130140, filed on Nov. 7, 2023.

(30) Foreign Application Priority Data
Nov. 7, 2022 (WO) PCT/CN2022/130229

(51) Int. Cl. A61K 31/4172; A61P 9/14; A61P 39/06

(52) U.S. Cl. CPC A61K 31/4172; A61P 9/14; A61P 39/06

(57) ABSTRACT

The present invention provides novel methods for anti-aging, especially ameliorating or preventing vascular endothelial damage induced by progerin in a mammal, comprising administration to the mammal a composition comprising an effective amount of ergothioneine (EGT) or a pharmaceutically acceptable salt, acid, ester, analog or derivative thereof. The present invention also provides a composition comprising an effective amount of ergothioneine (EGT) or a pharmaceutically acceptable salt, acid, ester, analog or derivative thereof, for anti-aging, especially ameliorating or preventing vascular endothelial damage induced by progerin in a mammal.

### CROSS REFERENCE TO RELATED APPLICATION

This application is a continuation application of International Patent Application No. PCT/CN2023/130140, filed on Nov. 7, 2023, which claims the priority of the International Patent Application No. PCT/CN2022/130229, filed on Nov. 7, 2022, the contents of all of which are incorporated herein by reference in their entirety.

### SEQUENCE LISTING

The contents of the electronic XML sequence listing (File Name: EGT-19200191.xml; Size: 3 Kilobytes; and Date of Creation: Jul. 18, 2025) are herein incorporated by reference in entirety.

### BACKGROUND OF THE INVENTION

### Technical Field

The present invention generally relates to compositions and methods for anti-aging, especially ameliorating or preventing vascular endothelial damage in a mammal, in particular through inhibiting progerin.

### Background Technology

Progerin is a truncated version of the lamin A protein. Progerin is most often generated by a single nucleotide polymorphism (C1824T) in the gene that codes for Lamin A. This mutation activates a cryptic splice site and gives rise to a form of lamin A with a deletion of 50 amino acids near the C-terminus. Lamin A constitutes a major structural component of the lamina, a scaffold of proteins found inside the nuclear membrane of a cell. Progerin protein aberrantly accumulates in the nuclear membrane, preventing at least some of the normal scaffolding functions of lamin A, which in turn interferes with multiple processes in the nucleus, including chromatin organization, heterochromatin formation, the DNA-damage response, cell cycle, gene transcription, and telomere maintenance. Progerin interacts with cell environment and causes changes in the location and level of chromatin remodeling factors, transcription factors, DNA repairing factors, factors connected with the nuclear lamina. All of these changes influence cell hyperproliferation and cause an arrest of the cell, apoptosis, resulting in cellular senescence, dysfunction of tissue and organs.

Recently, it has been reported that progerin alters endothelial cell function in mouse models in vivo, causing impaired mechanotransduction and a reduction of the atheroprotective endothelial nitric oxide synthase activity (Osmanagic-Myers, S., et al., Endothelial progerin expression causes cardiovascular pathology through an impaired mechanoresponse. 2019. 129(2): p. 531-545). These alterations could participate in the severe contractile impairment observed in Hutchinson-Gilford progeria syndrome (HGPS) patients (del Campo, L., et al., Vascular Smooth Muscle Cell-Specific Progerin Expression Provokes Contractile Impairment in a Mouse Model of Hutchinson-Gilford Progeria Syndrome that Is Ameliorated by Nitrite Treatment. 2020. 9(3): p. 656). Expression of progerin has been reported in atherosclerotic coronary arteries from aging individuals (Ashapkin, V. V., et al., Are There Common Mechanisms Between the Hutchinson-Gilford Progeria Syndrome and Natural Aging? 2019. 10: p. 455; Olive, M., et al., Cardiovascular pathology in Hutchinson-Gilford progeria: correlation with the vascular pathology of aging. 2010. 30(11): p. 2301-2309). Additionally, according to previous study, progerin overexpression in endothelial cells recapitulates some features of aging-associated endothelial cell dysfunction, including a proinflammatory phenotype and oxidative stress together with persistent DNA damage, increased cell cycle arrest protein expression and cellular senescence (Bidault, G., et al., Progerin expression induces inflammation, oxidative stress and senescence in human coronary endothelial cells. 2020. 9(5): p. 1201) and the HGPS and physiological aging could share a common cellular and molecular basis at least lamin A participates in the aging process in healthy individuals (Scaffidi, P. and T. J. S. Misteli, Lamin A-dependent nuclear defects in human aging. 2006. 312(5776): p. 1059-1063).

Compositions and methods are needed against the negative effects of progerin on aging and especially vascular endothelial damage or pro-inflammatory cytokine.

### SUMMARY OF THE PRESENT INVENTION

In a first aspect, the present invention provides a method for anti-aging in a mammal, the method comprising administration to the mammal a composition comprising an effective amount of ergothioneine or a pharmaceutically acceptable salt, acid, ester, analog or derivative thereof.

In some embodiments, the anti-aging is achieved through inhibiting progerin.

In some embodiments, the method is used for ameliorating or preventing vascular endothelial damage induced by progerin.

In some embodiments, the vascular endothelial damage comprises: cell inflammation, arrest of cells, apoptosis, cellular senescence, dysfunction of tissue and organs; the ameliorating or preventing vascular endothelial damage comprises decreasing pro-inflammatory cytokine selected from IL-1α, IL-1β, IL-6, TNF-α.

In some embodiments, the mammal is human.

In some embodiments, the composition is prepared in a form of food, drink, nutritional supplement, cosmetic product, or pharmaceutical composition.

In some embodiments, the administration is through various routes selected from oral administration, intravenous injection, intramuscular injection, intraperitoneal injection, topical application, or sublingual application.

In some embodiments, the ergothioneine or a pharmaceutically acceptable salt, acid, ester, analog or derivative thereof is administered at a daily dose of 1-2000 mg. In some embodiments, the ergothioneine or a pharmaceutically acceptable salt, acid, ester, analog or derivative thereof is administrated at a daily dose of 1-1500 mg, 2-1000 mg, 2-500 mg, 3-100 mg, or 5-50 mg. In some embodiments, the administration is at least once a day or more times a day. In some embodiments, the composition is administered in divided doses or a single dose.

In some embodiments, the ergothioneine or a pharmaceutically acceptable salt, acid, ester, analog or derivative thereof is formulated in solutions, liquid suspensions, parenteral solutions, injections, tablets, pills, granules, powders, films, (micro)capsules, aerosols, tonics, syrups, beverages, nourishments, snacks, bars, gums, sugars, a facial mask composition, a functionalized cream composition, a functionalized essence, a skin care composition, a make-up composition or a functionalized food composition.

In a second aspect, the present invention provides a composition comprising an effective amount of ergothioneine or a pharmaceutically acceptable salt, acid, ester, analog or derivative thereof, for anti-aging in a mammal.

In some embodiments, the anti-aging is achieved through inhibiting progerin.

In some embodiments, the composition is used for ameliorating or preventing vascular endothelial damage induced by progerin

In some embodiments, the vascular endothelial damage comprises: cell inflammation, arrest of cells, apoptosis, cellular senescence, dysfunction of tissue and organs; the ameliorating or preventing vascular endothelial damage comprises decreasing pro-inflammatory cytokine selected from IL-1α, IL-1β, IL-6, TNF-α.

In some embodiments, the mammal is human.

In some embodiments, the composition is prepared in a form of food, drink, nutritional supplement, cosmetic product, or pharmaceutical composition.

In some embodiments, the ergothioneine or a pharmaceutically acceptable salt, acid, ester, analog or derivative thereof is administered at a daily dose of 1-2000 mg. In some embodiments, the ergothioneine or a pharmaceutically acceptable salt, acid, ester, analog or derivative thereof is administrated at a daily dose of 1-1500 mg, 2-1000 mg. 2-500 mg, 3-100 mg, or 5-50 mg.

In some embodiments, the ergothioneine or a pharmaceutically acceptable salt, acid, ester, analog or derivative thereof is formulated in solutions, liquid suspensions, parenteral solutions, injections, tablets, pills, granules, powders, films, (micro)capsules, aerosols, tonics, syrups, beverages, nourishments, snacks, bars, gums, sugars, a facial mask composition, a functionalized cream composition, a functionalized essence, a skin care composition, a make-up composition or a functionalized food composition.

In a third aspect, the present invention provides use of a composition for preparing food, drink, nutritional supplement, cosmetic product, or pharmaceutical composition for anti-aging in a mammal, the composition comprises an effective amount of ergothioneine or a pharmaceutically acceptable salt, acid, ester, analog or derivative thereof.

In some embodiments, the anti-aging is achieved through inhibiting progerin.

In some embodiments, the composition is used for ameliorating or preventing vascular endothelial damage induced by progerin.

In some embodiments, the vascular endothelial damage comprises: cell inflammation, arrest of cells, apoptosis, cellular senescence, dysfunction of tissue and organs; the ameliorating or preventing vascular endothelial damage comprises decreasing pro-inflammatory cytokine selected from IL-1α, IL-1β, IL-6, TNF-α.

In some embodiments, the ergothioneine or a pharmaceutically acceptable salt, acid, ester, analog or derivative thereof is administered at a daily dose of 1-2000 mg. In some embodiments, the ergothioneine or a pharmaceutically acceptable salt, acid, ester, analog or derivative thereof is administrated at a daily dose of 1-1500 mg, 2-1000 mg. 2-500 mg, 3-100 mg, or 5-50 mg.

In some embodiments, the ergothioneine or a pharmaceutically acceptable salt, acid, ester, analog or derivative thereof is formulated in solutions, liquid suspensions, parenteral solutions, injections, tablets, pills, granules, powders, films, (micro)capsules, aerosols, tonics, syrups, beverages, nourishments, snacks, bars, gums, sugars, a facial mask composition, a functionalized cream composition, a functionalized essence, a skin care composition, a make-up composition or a functionalized food composition.

In a fourth aspect, the present invention provides a method for inhibiting progerin in a mammal, the method comprising administration to the mammal a composition comprising an effective amount of ergothioneine or a pharmaceutically acceptable salt, acid, ester, analog or derivative thereof.

In some embodiments, the method is used for anti-aging.

In some embodiments, the method is used for ameliorating or preventing vascular endothelial damage induced by progerin.

In some embodiments, the vascular endothelial damage comprises: cell inflammation, arrest of cells, apoptosis, cellular senescence, dysfunction of tissue and organs; the ameliorating or preventing vascular endothelial damage comprises decreasing pro-inflammatory cytokine selected from IL-1α, IL-1β, IL-6, TNF-α.

In some embodiments, the mammal is human.

In some embodiments, the composition is prepared in a form of food, drink, nutritional supplement, cosmetic product, or pharmaceutical composition.

In some embodiments, the administration is through various routes selected from oral administration, intravenous injection, intramuscular injection, intraperitoneal injection, topical application, or sublingual application.

In some embodiments, the ergothioneine or a pharmaceutically acceptable salt, acid, ester, analog or derivative thereof is administered at a daily dose of 1-2000 mg. In some embodiments, the ergothioneine or a pharmaceutically acceptable salt, acid, ester, analog or derivative thereof is administrated at a daily dose of 1-1500 mg, 2-1000 mg, 2-500 mg, 3-100 mg, or 5-50 mg. In some embodiments, the administration is at least once a day or more times a day. In some embodiments, the composition is administered in divided doses or a single dose.

In some embodiments, the ergothioneine or a pharmaceutically acceptable salt, acid, ester, analog or derivative thereof is formulated in solutions, liquid suspensions, parenteral solutions, injections, tablets, pills, granules, powders, films, (micro)capsules, aerosols, tonics, syrups, beverages, nourishments, snacks, bars, gums, sugars, a facial mask composition, a functionalized cream composition, a functionalized essence, a skin care composition, a make-up composition or a functionalized food composition.

In a fifth aspect, the present invention provides a composition comprising an effective amount of ergothioneine or a pharmaceutically acceptable salt, acid, ester, analog or derivative thereof, for inhibiting progerin in a mammal.

In some embodiments, the composition is used for anti-aging.

In some embodiments, the composition is used for ameliorating or preventing vascular endothelial damage induced by progerin

In some embodiments, the vascular endothelial damage comprises: cell inflammation, arrest of cells, apoptosis, cellular senescence, dysfunction of tissue and organs; the ameliorating or preventing vascular endothelial damage comprises decreasing pro-inflammatory cytokine selected from IL-1α, IL-1β, IL-6, TNF-α.

In some embodiments, the mammal is human.

In some embodiments, the composition is prepared in a form of food, drink, nutritional supplement, cosmetic product, or pharmaceutical composition.

In some embodiments, the ergothioneine or a pharmaceutically acceptable salt, acid, ester, analog or derivative thereof is administered at a daily dose of 1-2000 mg. In some embodiments, the ergothioneine or a pharmaceutically acceptable salt, acid, ester, analog or derivative thereof is administrated at a daily dose of 1-1500 mg, 2-1000 mg, 2-500 mg, 3-100 mg, or 5-50 mg.

In some embodiments, the ergothioneine or a pharmaceutically acceptable salt, acid, ester, analog or derivative thereof is formulated in solutions, liquid suspensions, parenteral solutions, injections, tablets, pills, granules, powders, films, (micro)capsules, aerosols, tonics, syrups, beverages, nourishments, snacks, bars, gums, sugars, a facial mask composition, a functionalized cream composition, a functionalized essence, a skin care composition, a make-up composition or a functionalized food composition.

In a sixth aspect, the present invention provides use of a composition for preparing food, drink, nutritional supplement, cosmetic product, or pharmaceutical composition for inhibiting progerin in a mammal, the composition comprises an effective amount of ergothioneine or a pharmaceutically acceptable salt, acid, ester, analog or derivative thereof. In some embodiments, the composition is used for anti-aging.

In some embodiments, the composition is used for ameliorating or preventing vascular endothelial damage induced by progerin.

In some embodiments, the vascular endothelial damage comprises: cell inflammation, arrest of cells, apoptosis, cellular senescence, dysfunction of tissue and organs; the ameliorating or preventing vascular endothelial damage comprises decreasing pro-inflammatory cytokine selected from IL-1α, IL-1β, IL-6, TNF-α.

In some embodiments, the ergothioneine or a pharmaceutically acceptable salt, acid, ester, analog or derivative thereof is administered at a daily dose of 1-2000 mg. In some embodiments, the ergothioneine or a pharmaceutically acceptable salt, acid, ester, analog or derivative thereof is administrated at a daily dose of 1-1500 mg, 2-1000 mg, 2-500 mg, 3-100 mg, or 5-50 mg.

In some embodiments, the ergothioneine or a pharmaceutically acceptable salt, acid, ester, analog or derivative thereof is formulated in solutions, liquid suspensions, parenteral solutions, injections, tablets, pills, granules, powders, films, (micro)capsules, aerosols, tonics, syrups, beverages, nourishments, snacks, bars, gums, sugars, a facial mask composition, a functionalized cream composition, a functionalized essence, a skin care composition, a make-up composition or a functionalized food composition.

These and other features, aspects, and advantages of the present invention will become better understood with reference to the following description and appended claims.

### BRIEF DESCRIPTION OF THE FIGURES

FIG. 1 shows normalized Lamin mRNA expression in coronary artery endothelial cells of different groups.

FIG. 2 shows relative mRNA expression of the pro-inflammatory cytokines in different groups.

### DETAILED DESCRIPTION OF THE INVENTION

In the Summary Section above and the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Ergothioneine (EGT) is a sulfhydryl-containing derivative of the histidine amino acid with a powerful antioxidant capability. Studies show that it can accumulate in organs, cells and secretions that are exposed to high levels of oxidative stress and inflammation (Tang, R., I. Cheah, and T. Yew, Distribution and accumulation of dietary ergothioneine and its metabolites in mouse tissues. Sci Rep 8: 1601. 2018). EGT has also been shown to inhibit IL-1β-induced expression of vascular and intracellular adhesion molecules in endothelial cells (Anticoli, S., et al., 7-ketocholesterol and 5,6-secosterol modulate differently the stress-activated mitogen-activated protein kinases (MAPKs) in liver cells. 2010. 222(3): p. 586-595).

Given that the accumulation of progerin could cause damage to the vascular endothelial cell, while EGT acts as a good physiological cytoprotectant, the inventors of this invention has tested the inhibition effect of EGT on progerin induced damage. In the experiment, aging mice model were treated with EGT or not. The results of present work showed that EGT could inhibit the expression of progerin in coronary artery endothelial cells. Additionally, EGT could decrease the inflammatory factors, thus mitigate the damage induce by progerin. These results suggest that EGT possesses good potential to be further developed into a promising candidate to ameliorate vascular endothelial damage induced by progerin and provide benefits for mitigating atherosclerosis.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” and their conjugations, refer to a situation wherein said terms are used in their non-limiting sense to mean that items following the word are included, but items not specifically mentioned are not excluded. It also encompasses the more limiting verb ‘to consist essentially of’ and “to consist of”.

As used herein, the term “effective amount” refers to the amount required to achieve the effect as taught herein. The specific effective dose level for any particular subject will depend upon a variety of factors including the conditions being treated and the severity of the conditions; the specific composition employed; the age, body weight, general health, sex and diet of the subject; the time of administration, route of administration, and rate of excretion of ergothioneine (EGT) or its analog or its derivatives employed; the duration of the treatment; and like factors well known in the medical arts. For example, it is well known within the skill of the art to start doses of the compound at levels lower than those required to achieve the desired effect and to gradually increase the dosage until the desired effect is achieved.

One of skill in the art recognizes that an amount may be considered “effective” even if the condition is not totally eradicated or prevented, but it or its symptoms and/or effects are improved or alleviated partially in the subject.

As used herein, the term “pharmaceutically acceptable” means pharmaceutically, physiologically, alimentarily, and/or nutritionally acceptable, and refers to those compositions or combinations of agents, materials, or compositions, and/or their dosage forms, which are within the scope of sound medical judgment, suitable for use in contact with the tissues of human beings and animals without excessive toxicity, irritation, allergic response, or other problem or complication, commensurate with a reasonable benefit/risk ratio.

As used herein, the term “mammal” or “subject” may be used interchangeably to refer to any animal to which the presently disclosed methods and compositions may be applied or administered. The animal may have an illness or other disease, but the animal does not need to be sick to benefit from the presently disclosed methods and compositions. As such any animal may apply the disclosed combinations, compositions or kits, or be a recipient of the disclosed methods. “Mammal” includes, without limitation, mice, rats, rabbits, guinea pigs, dogs, cats, sheep, goats, cows, horses, primates, such as monkeys, chimpanzees, and apes, and, in particular, humans. Although the animal subject is preferably a human, the methods and compositions of the invention have application in veterinary medicine.

The dosage of ergothioneine or a pharmaceutically acceptable salt, acid, ester, analog or derivative thereof and/or composition comprising the same may range broadly, depending upon the desired effects and the indication. The daily dosage regimen for an adult human patient may be, for example, an oral dose of between 0.01 mg and 3000 mg of ergothioneine or its analog or derivative, preferably between 1 mg and 700 mg, e.g., 5 to 200 mg, or between about 0.1 mg and about 1,000 mg of ergothioneine or its analog or derivative per kg of body weight of the subject. The dosage may be a single one or a series of two or more given in the course of one or more days, as is needed by the subject. In some embodiments, the compounds are administered for a period of time, for example for a week or more, or for months or years.

As used herein, the term “administration” refers to the process of delivering a disclosed composition or active ingredient to a subject The compositions of the invention can be administered in a variety of ways, including orally, intragastrically, and parenterally (e.g., intravenous and intraarterial as well as other suitable parenteral routes), and the like.

As used herein, a “parenteral solution” refers to a solution that can be administered elsewhere in the body than the mouth and alimentary canal. It is not delivered via the intestinal tract. For example, parenteral solution can be delivered intravenously.

As used herein, a “tonic” refers to a medicinal substance taken to give a feeling of vigor or well-being.

As used herein, a “syrup” refers to a thick sticky liquid derived from a sugar-rich plant, for example, sugar cane, corn, and maple.

Multiple techniques of administering a composition exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections.

“Intraperitoneal” as used here means within or administered through the peritoneum. The peritoneum is a thin, transparent membrane that lines the walls of the abdominal (peritoneal) cavity and contains/encloses the abdominal organs such as the stomach and intestines.

As used herein, “sublingual” refers to situated or applied under the tongue.

A “functionalized cream composition” includes a cream composition that has a potentially positive effect on health beyond basic nutrition.

An “essence” includes an extract or concentrate obtained from a particular plant or other matter and used for flavoring or scent. A “functionalized essence” includes an essence that has a potentially positive effect on health beyond basic nutrition.

A “functionalized food composition” includes a food composition that has a potentially positive effect on health beyond basic nutrition.

In some embodiments, ergothioneine or a pharmaceutically acceptable salt, acid, ester, analog or derivative thereof and/or composition comprising the same can ameliorate or prevent vascular endothelial damage.

“Vascular endothelial damage” herein comprises, but is not limited to, cell inflammation, arrest of cells, apoptosis, cellular senescence, dysfunction of tissue and organs.

“Cell inflammation” here refers to inflammation of cells, such as endothelial cells.

“Arrest of cells” here refers to cell cycle arrest. In mammals, the cell cycle consists of four successive phases: in the S phase, the cell replicates its DNA, while in mitosis (M phase), the cell partitions its replicated DNA into two daughter cells. Two gap phases (G1 and G2) separate DNA replication from mitosis; G1 extends from mitosis to the next round of DNA replication, while G2 is the gap between S and the next M phase. Cell cycle arrest is a state where cells are no longer involved in the processes surrounding duplication and division.

“Apoptosis” is a form of programmed cell death that occurs in multicellular organism. Biochemical events lead to characteristic cell changes (morphology) and death. These changes include blebbing, cell shrinkage, nuclear fragmentation, chromatin condensation, DNA fragmentation, and mRNA decay. The initiation of apoptosis is tightly regulated by activation mechanisms, because once apoptosis has begun, it inevitably leads to the death of the cell.

“Cellular senescence” is a phenomenon characterized by the cessation of cell division. Cellular senescence can be initiated by a wide variety of stress inducing factors. These stress factors include both environmental and internal damaging events, abnormal cellular growth, oxidative stress, autophagy factors, among many other things.

In some embodiments, ergothioneine or a pharmaceutically acceptable salt, acid, ester, analog or derivative thereof and/or composition comprising the same can decrease levels of progerin, lamin A, and lamin C in mammal cells. In some embodiments, the cells comprise vascular endothelial cells.

Lamin A and lamin C are two major proteins produced from the LMNA gene, which provides instructions for making several slightly different proteins called lamins. Lamin A and lamin C are made in most of the body's cells. These proteins are made up of a nearly identical sequence of protein building blocks (amino acids). The small difference in the sequence makes lamin A longer than lamin C.

Lamins A and C are structural proteins called intermediate filament proteins. Intermediate filaments provide stability and strength to cells. Lamins A and C are supporting (scaffolding) components of the nuclear envelope, which is a structure that surrounds the nucleus in cells. Specifically, these proteins are located in the nuclear lamina, a mesh-like layer of intermediate filaments and other proteins that is attached to the inner membrane of the nuclear envelope. The nuclear envelope regulates the movement of molecules into and out of the nucleus. Lamins A and C are also found inside the nucleus, and researchers believe the proteins may play a role in regulating the activity (expression) of certain genes.

In some embodiments, ergothioneine or a pharmaceutically acceptable salt, acid, ester, analog or derivative thereof and/or composition comprising the same can decrease pro-inflammatory cytokine.

“Pro-inflammatory cytokine” herein comprises, but is not limited to IL-1α, IL-1β, IL-6, TNF-α.

Any titles or subheadings used herein are for organization purposes and should not be used to limit the scope of embodiments disclosed herein.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

All publications and patent applications cited in this specification are herein incorporated by reference as if each individual publication or patent application were specifically and individually indicated to be incorporated by reference.

### EXAMPLES

### Example 1

18 C57BJ/6 male mice aged about 8 weeks, were randomly assigned to three study groups (GA, GB, GC). The total experimental time was 56 weeks, and the control group was consisted of 12 animals, and all of the animals were maintained in an animal facility room under controlled environmental conditions (22±1° C., 12 h-light/12 h dark cycle, free access to food and water). All procedures were carried out in accordance with institutional policies on experimental animal handling.

Treatment details of the individual study groups were: Group A (GA): euthanized at the age of 10 weeks; Group B (GB): euthanized at the age of 56 weeks without EGT treated; Group C (GC): euthanized at the age of 56 weeks with EGT treated every day.

For all the euthanized mice, formalin-fixed paraffin embedded 3 μm sections were prepared from the tissues of coronary artery, and were snap-frozen and stored at −80° C.

Taqman quantitative real-time PCR was used for detection of progerin mRNA. Messenger RNA was extracted using RNeasy Mini Kit Plus (Qiagen, Valencia, CA). The Taqman probe was as described by Rodriguez et al. (Rodriguez, S., et al., Increased expression of the Hutchinson Gilford progeria syndrome truncated lamin A transcript during cell aging. 2009. 17(7): p. 928-937). The forward primer was 5′-ACTGCAGCAGCTCGGGG-3′ (SEQ ID NO: 1), and the reverse primer was 5′-GG CTCTGGGCTCCTGAGCC-3′ (SEQ ID NO: 2). Taqman PCR enzyme mix as well as primers and probe for actin as internal control were purchased from Life Technologies (Grand Island, NY).

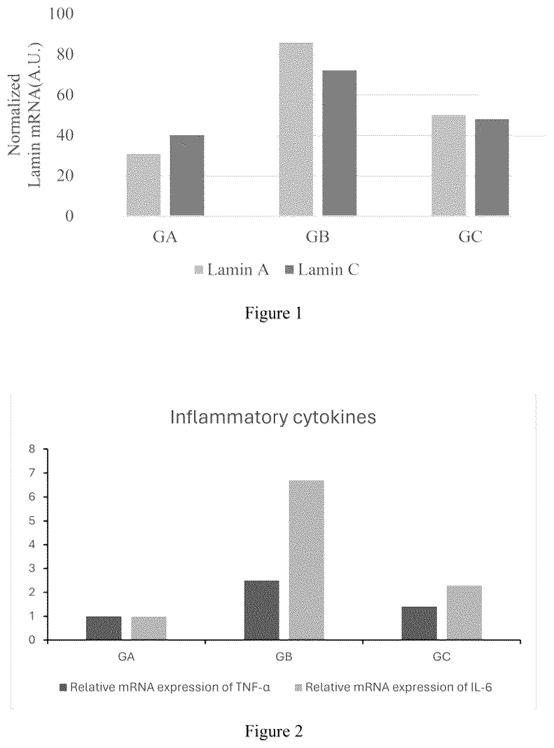

FIG. 1 shows normalized Lamin mRNA expression in coronary artery endothelial cells of different groups. Taqman quantitative real-time PCR analysis showed significant increase in the mRNA expression of progerin in coronary artery endothelial cells in 56-week-old mice compared to that of 10-week-old mice, while an obvious decrease in the mRNA expression of progerin treated with EGT compared to that of without EGT treated, as well as the decrease in the mRNA of lamin A and lamin C. Lamin A and Lamin C are critical structural proteins of the nuclear lamina. Abnormal increase (such as overexpression) of Lamin A/C usually leads to nuclear structural disruption, DNA damage, mitochondrial dysfunction, oxidative stress, cell cycle dysregulation, and inflammatory activation, which will lead to vascular endothelial damage and aging. In the invention, the administration of EGT can reduce lamin A and lamin C, thus inhibiting vascular endothelial damage and aging.

### Example 2

Endothelial cell inflammation and senescence have been shown to increase susceptibility to atherosclerosis during normal aging (Donato, A. J., et al., Cellular and molecular biology of aging endothelial cells. 2015. 89: p. 122-135) and expression of progerin has been reported in atherosclerotic coronary arteries from aging individuals (Ashapkin, V. V., et al., Are There Common Mechanisms Between the Hutchinson-Gilford Progeria Syndrome and Natural Aging? 2019. 10: p. 455; Olive, M., et al., Cardiovascular pathology in Hutchinson-Gilford progeria: correlation with the vascular pathology of aging. 2010. 30(11): p. 2301-2309). So, experiments are designed to detect the pro-inflammatory cytokines to confirm whether EGT could ameliorate the inflammation induced by progerin.

The details of animal treatment information are described above in Example 1. Quantification of pro-inflammatory cytokines was performed on 56-week-old animal serum using ELISA Array kit.

FIG. 2 shows relative mRNA expression of the pro-inflammatory cytokines in different groups. The ELISA assay showed that the pro-inflammatory cytokines IL-1α, IL-1β, IL-6, and TNF-α were significantly higher in 56-week-old mice compared to that of 10-week-old. In addition, an obvious decrease of the pro-inflammatory cytokines in the mice being pretreated with EGT compared to those of without EGT treatment. The results suggest that the EGT could alleviate the inflammation induced by progerin.

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications as fall within the scope of the appended claims.

What is claimed is:

1. A method of ameliorating or preventing vascular endothelial damage for anti-aging in a mammal, the method comprising administration to the mammal a composition comprising an effective amount of ergothioneine or a pharmaceutically acceptable salt, acid, ester, analog or derivative thereof.

2. The method of claim 1, wherein the anti-aging is achieved through inhibiting progerin.

3. The method of claim 1, wherein the method is used for ameliorating or preventing vascular endothelial damage induced by progerin.

4. The method of claim 3, wherein the vascular endothelial damage comprises: cell inflammation, arrest of cells, apoptosis, cellular senescence, dysfunction of tissue and organs; the ameliorating or preventing vascular endothelial damage comprises decreasing pro-inflammatory cytokine selected from IL-1α, IL-1β, IL-6, TNF-α.

5. The method of claim 1, wherein the mammal is human.

6. The method of claim 1, wherein the composition is prepared in a form of food, drink, nutritional supplement, cosmetic product, or pharmaceutical composition.

7. The method of claim 1, wherein the administration is through various routes selected from oral administration, intravenous injection, intramuscular injection, intraperitoneal injection, topical application, or sublingual application.

8. The method of claim 1, wherein the ergothioneine or a pharmaceutically acceptable salt, acid, ester, analog or derivative thereof is administered at a daily dose of 1-2000 mg.

9. The method of claim 1, wherein the ergothioneine or a pharmaceutically acceptable salt, acid, ester, analog or derivative thereof is formulated in solutions, liquid suspensions, parenteral solutions, injections, tablets, pills, granules, powders, films, (micro)capsules, aerosols, tonics, syrups, beverages, nourishments, snacks, bars, gums, sugars, a facial mask composition, a functionalized cream composition, a functionalized essence, a skin care composition, a make-up composition or a functionalized food composition.

10. The method of claim 1, wherein the administration is at least once a day or more times a day.

11. A method for inhibiting progerin in a mammal, the method comprising administration to the mammal a composition comprising an effective amount of ergothioneine or a pharmaceutically acceptable salt, acid, ester, analog or derivative thereof.

12. The method of claim 11, wherein the method is used for anti-aging.

13. The method of claim 11, wherein the method is used for ameliorating or preventing vascular endothelial damage induced by progerin.

14. The method of claim 13, wherein the vascular endothelial damage comprises: cell inflammation, arrest of cells, apoptosis, cellular senescence, dysfunction of tissue and organs; the ameliorating or preventing vascular endothelial damage comprises decreasing pro-inflammatory cytokine selected from IL-1α, IL-1β, IL-6, TNF-α.

15. The method of claim 11, wherein the mammal is human.

16. The method of claim 11, wherein the composition is prepared in a form of food, drink, nutritional supplement, cosmetic product, or pharmaceutical composition.

17. The method of claim 11, wherein the administration is through various routes selected from oral administration, intravenous injection, intramuscular injection, intraperitoneal injection, topical application, or sublingual application.

18. The method of claim 11, wherein the ergothioneine or a pharmaceutically acceptable salt, acid, ester, analog or derivative thereof is administered at a daily dose of 1-2000 mg.

19. The method of claim 11, wherein the ergothioneine or a pharmaceutically acceptable salt, acid, ester, analog or derivative thereof is formulated in solutions, liquid suspensions, parenteral solutions, injections, tablets, pills, granules, powders, films, (micro)capsules, aerosols, tonics, syrups, beverages, nourishments, snacks, bars, gums, sugars, a facial mask composition, a functionalized cream composition, a functionalized essence, a skin care composition, a make-up composition or a functionalized food composition.

20. The method of claim 11, wherein the administration is at least once a day or more times a day.


SEQUENCE LISTING (2 sequences)

SEQ ID NO: 1 - DNA, 17 bp, synthetic construct
  source 1..17: mol_type = other DNA
actgcagcagctcgggg

SEQ ID NO: 2 - DNA, 19 bp, synthetic construct
  source 1..19: mol_type = other DNA
ggctctgggctcctgagcc